(19) United States
(12) Patent Application Publication
(10) Pub. No.: US 2006/0282119 A1
(43) Pub. Date: Dec. 14, 2006

(54) Protective device for a suture

(75) Inventor: Joel E. Perchik, Jackson, TN (US)

Correspondence Address: Lloyd McAulay;Reed Smith LLP, 599 Lexington Avenue, New York, NY, 10022-7650, US

(73) Assignee: AngioDynamics, Inc.

(21) Appl. No.: 11/149,917

(22) Filed: Jun. 10, 2005

(51) Int. Cl. A61B 17/04

(52) U.S. Cl. 606/232

(57) ABSTRACT

A disc for holding a medical suture on a patient's skin is modified by providing first and second orthogonal grooves in a domed upper surface. Through openings in one of the grooves permits the suture to be tied within that groove so that the suture has a knot contained within the groove and thus below the domed upper surface. The second groove, preferably orthogonal to the first groove, permits a scalpel to be readily inserted along the second groove to traverse the first groove and cut the suture when the suture is to be removed.

### BACKGROUND OF THE INVENTION

This invention relates in general to a device to be used in connection with the suturing of a patient. The device serves the purpose of protecting the skin of the patient and maintaining the integrity of the suture.

When a suture is made on a patient's skin, various potential problems may occur. The suture may be partially incorporated into the skin as the skin heals. The tying of the suture provides a small force over a very small area creating substantial pressure between the suture and the patient's skin. It is known to use a small button like disc to hold the suture on the patient's skin to overcome these problems.

However, the suture knot stands up from the disc and may be abraded and sometimes can become unknotted. When the suture is to be removed, medical personnel have to be pick the suture off the disc to cut the suture with a scalpel.

It is known to use a small disc such as a button with two openings that extend through the buttons so that a suture can be threaded through the openings and tied on top of the button thereby separating the suture knot from the patient's skin. This provides a degree of patient comfort and minimizes the risk of having the knot embedded in the skin.

Most patients are ambulatory and generally go about their own business during the time that a suture wound is healing. This risks having the knot suture abraded and possibly untied.

Although a bandage is usually placed over the suture knot, it is often removed. The disc with a suture knot tends to create a profile that is not smooth and sometimes snags when putting on clothing.

The suture is removed by placing a scalpel under the suture and cutting upward. This technique of putting a blade under the fairly tightly tied knot pulls on the patient and requires care on the part of medical personnel.

A major purpose of this invention to provide a protective disc which is improved so that the cutting of the suture will be facilitated, providing an easier technique for the medical personnel.

It is a further object of this invention to provide a protective device to be used with a suture which will minimize accidental unknotting of the suture and to provide an improved profile for the protective disc so as to minimize snagging of the exposed suture during patient movement.

It is a related object of this invention to obtain these objectives while retaining the advantages of the presently known disc.

### BRIEF DESCRIPTION

In brief, a preferred embodiment involves a small disc having first and second grooves along substantially perpendicular diameters on the top surface of the protective device. In a first one of the grooves, there are two openings through the disc to permit suture thread to be passed through and tied along the top of the disc. When tied, the suture thread and the suture knot sit in that first groove. This reduces the extent to which the suture knot is subject to abrading forces that rub across the suture. It also reduces the risk of the knot snagging when clothing is put on over the suture.

The second groove provides a track for the scalpel when cutting the suture. When the suture is to be removed, a cut made by a scalpel that is inserted along the second groove serves to sever the suture and allow its removal.

The upper surface of the disc is domed to provide a smoother profile that together with the recessed knot provides less discomfort for the wearer in pulling clothing over the bandaged disc and suture.

### BRIEF DESCRIPTION OF THE FIGURES

FIG. 1 is a perspective view of a preferred embodiment of this invention.

FIG. 2 is a top view of the FIG. 1 device.

FIG. 3 is a first side view of the FIG. 2 device.

FIG. 4 is a second side view, orthogonal to the FIG. 3 view, of the FIG. 1 device.

FIG. 5 is a perspective view of a second embodiment of this invention.

FIG. 6 is a top view of the FIG. 5 device.

FIG. 7 is a first side view of the FIG. 5 device.

FIG. 8 is a second side view of the FIG. 5 device. The FIG. 8 is orthogonal to the FIG. 7 view.

### DESCRIPTION OF THE PREFERRED EMBODIMENTS

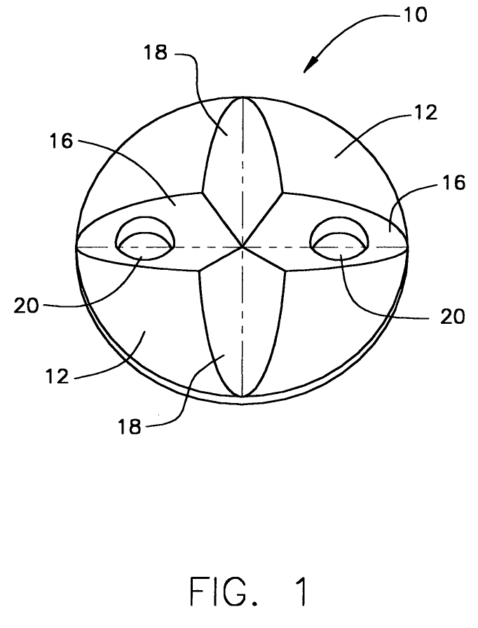

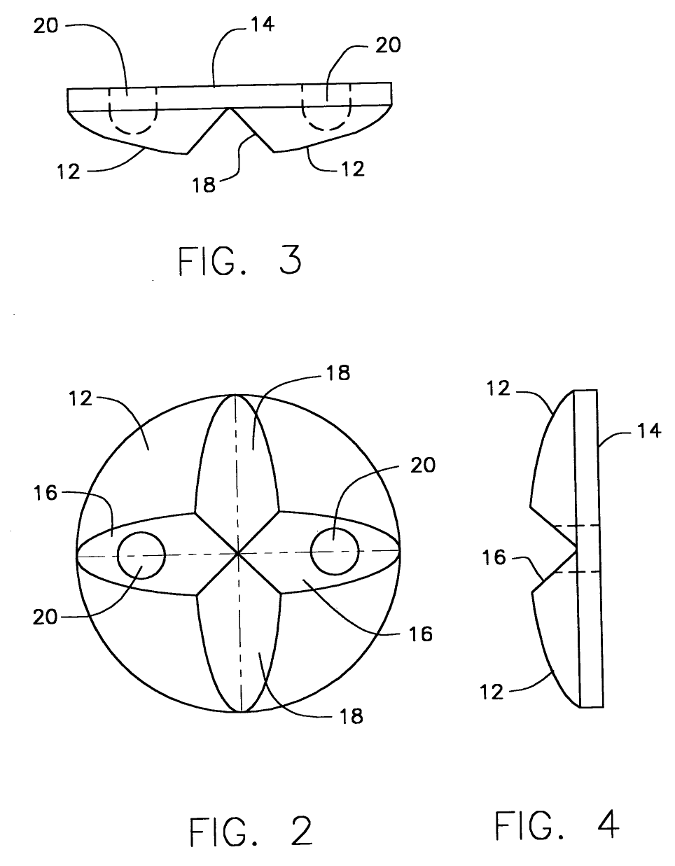

The preferred embodiment, as shown in FIGS. 1 through 4, is a disc-like protective device 10 having a domed upper surface 12 and a flat lower surface 14.

A first diametric groove 16 extends across the domed upper surface 12.

A second diametric groove 18 is perpendicular to the first groove 16 and extends across the domed upper surface 12.

Two openings 20 are located in the first groove 16 on either side of the intersection of the two grooves 16 and 18. These openings extend through the disc 10. These openings 20 allow a suture to pass through the openings 20 so that the suture can be tied into a knot.

The suture and, most importantly, the knot are recessed within the first groove 16 below the domed upper surface 12 thereby protecting the knot from abrasion and unknotting.

Recess of the suture knot into the groove 16 also provides a smooth profile in use so as to minimize snagging on clothing during donning of clothing or during patient movement.

The second groove 18 facilitates cutting the suture with a scalpel. The scalpel can be slid along the second groove 18 to come under the suture and cut the suture.

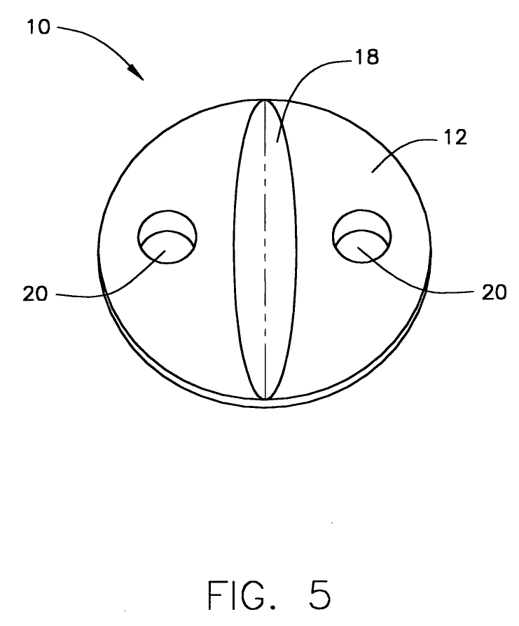

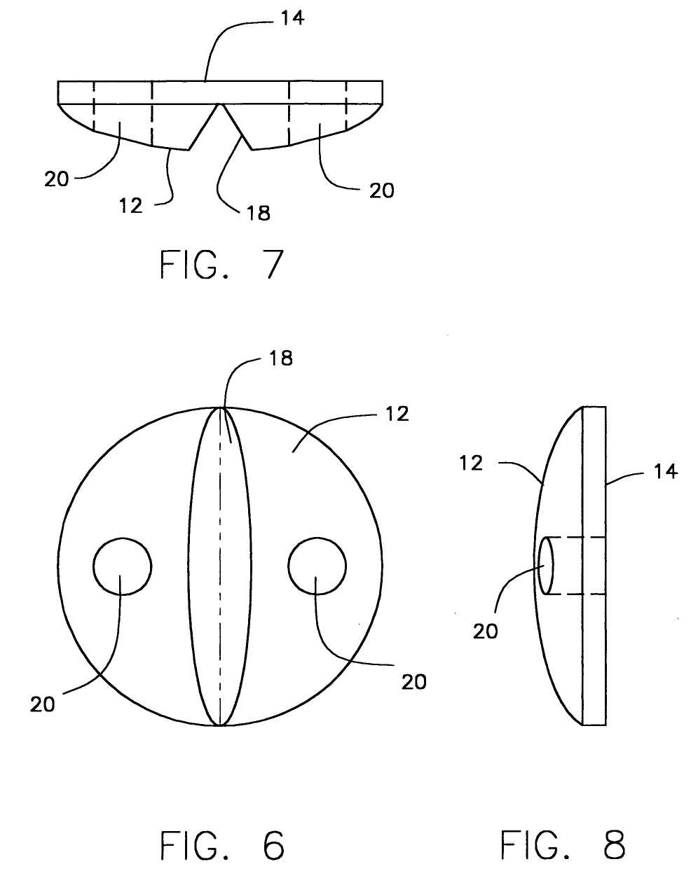

A second and less preferred embodiment of the invention is shown in FIGS. 5 through 8. The second embodiment includes the second groove 18 but not the first groove 16. Accordingly, in the second embodiment, the suture and knot are not recessed below the domed upper surface 12. However, the second groove 18 does continue to perform its function of providing a channel for the insertion of the scalpel under the suture in order to cut the suture when the suture is to be removed.

The suture is usually bandaged. However, the bandage is changed on a regular basis. Also patients often remove their bandages leaving the suture exposed. Further, the bandage may be removed to allow air into the area for healing purposes. So, although a bandage may be placed and provide temporary protection against abrasion, a bandage is not always in place over the wound.

The domed profile 12 provides a gradual smooth profile of the disc for patient comfort and non-snagging. In one embodiment, the radius of curvature of the dome is ⅜ inches on a disc that is 0.110 inches thick at the maximum.

Although the invention has been described in connection with the embodiments shown, there are certain variations which can be made by one skilled in the art without departing from the scope of this invention.

For example, it is clearly preferable to have the two grooves 16 and 18 in the preferred embodiment at right angles to one another. However, a major purpose of this invention is met by having the two grooves at something other than a right angle.

As another example, the grooves 16 and 18 can extend across the diameter of the disc or can terminate short of the edge of the disc.

What is claimed is:

1. A protective device to be used with a suture comprising:
a disc having an upper surface and a lower surface,
a first groove extending along said upper surface of said disc,
at least two openings extending through said disc, one on each side of said groove, sized to permit suture threads to pass there through.

2. The protective device of claim 1 wherein: said upper surface is domed.

3. The protective device of claim 1 wherein: said groove extends along the full extent of said upper surface of said disc.

4. The protective device of claim 2 wherein: said groove extends along the full extent of said upper surface of said disc

5. The protective device of claim 4 wherein: said disc is circular.

6. The protective device of claim 5 wherein: said groove extends along the full diameter of said circular disc.

7. A protective device to be used with a suture comprising:
a disc having an upper surface and a lower surface, first and second intersecting grooves extending along said upper surface of said disc,
at least two openings in said first groove, said openings positioned on opposite sides of the intersection between said grooves, said openings sized to permit suture threads to pass through.

8. The protective device of claim 7 wherein: said grooves extend along lines that are substantially perpendicular to each other.

9. The protective device of claim 7 wherein: said upper surface is domed.

10. The protective device of claim 8 wherein: said upper surface is domed.

11. The protective device of claim 8 wherein: said grooves extend along the full extent of said upper surface of said disc.

12. The protective device of claim 11 wherein: said disc is circular.

13. The protective device of claim 12 wherein: said groove extends along the full diameter of said circular disc.
